(19) United States
(12) Patent Application Publication
(10) Pub. No.: US 2014/0271828 A1
(43) Pub. Date: Sep. 18, 2014

(54) Pain Reliever Composition

(71) Applicant: Howard Rosen, Huntington Beach, CA (US)

(72) Inventor: Howard Rosen, Huntington Beach, CA (US)

(21) Appl. No.: 14/294,034

(22) Filed: Jun. 2, 2014

Related U.S. Application Data
(63) Continuation of application No. 13/479,998, filed on May 24, 2012, now abandoned.

(51) Int. Cl. A61K 36/886; A61K 36/185

(52) U.S. Cl. CPC A61K 36/886; A61K 36/185
USPC 424/450; 424/744

(57) ABSTRACT

A pain reliever comprised of dextrose, aloe vera concentrate, and some or all of the following ingredients: propylene glycol, caprylic/capric tryglicerides, sodium chloride (or acetic acid), a homeopathic anti-inflamatory extract, Dimethyl Sulfone (or Methylsulfonylmethane (MSM)), cetyl myristoleate, and, optionally, a pitcher plant extract. The resulting compositions are a water-based solution and two gel composition applied to the epidermis of mammals for relieving pain.

### CROSS-REFERENCE TO RELATED APPLICATION

This application claims the benefit of Continuation-in-part application Ser. No. 13/417,053 filed on Mar. 9, 2012, which is currently pending, and which claims the benefit of non-provisional patent application Ser. No. 13/295,010 filed on Nov. 11, 2011.

### FEDERALLY SPONSORED RESEARCH

Not Applicable

### SEQUENCE LISTING OR PROGRAM

Not Applicable

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

The invention relates to a pain reliever composition comprised of some or all of the following ingredients: dextrose, aloe vera concentrate, propylene glycol, caprylic/capric tryglicerides, sodium chloride (or acetic acid), a homeopathic anti-inflamatory extract, Dimethyl Sulfone (or methylsulfonylmethane (MSM)), cetyl myristoleate, and a pitcher plant extract.

Pain reliever compositions are known. For example, U.S. patent application Ser. No. 12/895,200 (US 2011/0076327 A1) by Lomax teaches herbal pain killer compositions, one of which comprises 50 mg each of the following ingredients formed into an approximately 600 mg tablet for oral administration to a mammal: Boswellia serrata, Tumeric, White Wilow, Harpagophytum Procumbens, Phellodendron Amurense, Paullinia Tomentosa, Milkberry, Mimosa Pudica, Lactuca Virosa, Naringen, 6-7 Dihydroxybergamottin, and Yerba mate.

Further, U.S. patent application Ser. No. 12/874,038 (US 2011/0117175 A1) by Rosenbaum teaches a pain reliever composition for medical procedures treatments comprising a sweet analgesic and a delivery vehicle, wherein the delivery vehicle is suitable for intra-oral delivery, and the sweet analgesic comprises sucrose, glucose, fructose, dextrose, maltodextrin, corn syrup, high fructose corn syrup, cyclamate, aspartame, sucralose, xylitol, cyclamate, stevia, brazzein, curculin, erythritol, glycyrrhizin, honey, luo han gua, mabinlin, monatin, miraculin, monellin, pentadia, thaumatin, acesulfame potassium, alitame, salt of aspartame-acesulfame, dulcin, glucin, neohyesperidin dihydrochalcone, neotame, P-4000, saccharin, or a combination thereof.

Finally, U.S. patent application Ser. No. 11/305,552 (US 2008/0102107 A1) by Lewellyn teaches a transdermal joint pain therapy composition comprising (a) from about 2.5% to about 15%, based on the total weight of said transdermal joint therapy composition, of glutamine; (b) from about 0.04% to about 0.5%, based on the total weight of said transdermal joint pain therapy composition, of hyaluronic acid; (c) from about 2.55 to about 10.0%, based on the total weight of said transdermal joint pain therapy composition, of methylsulfonylmethane; and (d) from about 70% to about 95%, based on the total weight of said transdermal joint pain therapy composition, of a transdermal delivery agent.

The objective of the present invention is to develop an alternate form of pain relief composition using different active ingredients and in different quantities that is applied to the epidermis of mammals.

### SUMMARY

The inventive pain reliever composition comprises a pain relief composition applied to the epidermis of mammals in form of a water-based solution and gels comprising dextrose and aloe vera concentrate, and further comprising some or all of the following ingredients: propylene glycol, caprylic/capric tryglicerides, sodium chloride (or acetic acid), a homeopathic anti-inflamatory extract, such as Traumeel®, Dimethyl Sulfone (or Methylsulfonylmethane (MSM)), cetyl myristoleate, lipoderm base, distilled water, and a pitcher plant extract, such as Sarapin.

The lipoderm base can be substituted by any of the following; (1) lecthicin or other fat soluble granules (2) PLO (Pluronic Lecithin Organogel) (3) Urea, (4) Oleic acid, (5) Liposomes, (6) Niosomes, or (7) Nanotechnology, namely, any one of the following chosen from a group consisting of nanocrystals, liposomes, nanoparticle-protein conjugates, magnetic nanoparticles, nanogels and biodegradable nanoparticlea. There are three preferred embodiments of the invention.

The first embodiment comprises aloe vera concentrate, propylene glycol, sterile water, and sodium chloride, in amounts ranging from 0.01% to 75% of the composition, but preferably comprising at least 5% anhydrous dextrose or 1% to 50% hypertonic saline (preferably 20%), at least 10% aloe vera concentrate, and at least 10% propylene glycol.

The second embodiment comprises aloe vera concentrate, propylene glycol, caprylic/capric triglycerides, ultrasound gel, and simple-gel (Hawkins), also in amounts ranging from 0.01% to 75% of the composition, but preferably comprising at least 5% anhydrous dextrose or 1% to 50% hypertonic saline (preferably 20%), at least 10% aloe vera concentrate, at least 10% propylene glycol, and at least 10% caprylic/capric triglycerides.

The third embodiment comprises anhydrous dextrose, aloe vera concentrate, ethoxy diglycol reagent, caprylic/capric triglycerides, lipoderm base, and cetyl myristoleat, in amounts ranging from 0.01% to 75%, but preferably comprising at least 20% anhydrous dextrose, at least 10% aloe vera concentrate, at least 10% caprylic/capric triglycerides, and at least 10% caprylic/capric triglycerides. Preferably, the third embodiment should further comprise at least 10% dimethyl sulfone or Methylsulfonylmethane (MSM), at least 10% pitcher plant extract, distilled water, and a homeopathic anti-inflammatory extract.

### DETAILED DESCRIPTION

The inventive pain reliever comprises anhydrous dextrose and aloe vera concentrate, and some or all of the following ingredients: propylene glycol, caprylic/capric tryglicerides, sodium chloride (or acetic acid), a homeopathic anti-inflamatory extract, such as Traumeel®, Dimethyl Sulfone (or Methylsulfonylmethane (MSM)), cetyl myristoleate, lipoderm base, distilled water, and, optionally, a pitcher plant extract, such as Sarapin. Each of the below-described embodiments are described in relation to a 100 gram composition.

In all embodiments, the Lipoderm base may be substituted by any of the following:
(1) lecthicin or other fat soluble granules in the range of 0.001 to 75% (w/w) of the composition, preferably 5%;
(2) PLO(Pluronic Lecithin Organogel) in the range of 0.001 to 75% of the composition in gel form, preferably 30%;
(3) Urea in the range of 1-50% of the composition, preferably 10%;
(4) Oleic acid in the range of 0.001 to 70% of the composition, preferably 5%;
(5) Liposomes in the range of 0.01% to 75% of the composition;
(6) Niosomes in the range of 0.01% to 75% of the composition; or
(7) Nanotechnology in the range of 0.01 to 75%, namely, any one of the following chosen from a group consisting of nanocrystals, liposomes, nanoparticle-protein conjugates, magnetic nanoparticles, nanogels and biodegradable nanoparticles.

Also, in all embodiments Anhydrous dextrose can be substituted in approximately the same concentraton by any of the following:
a. sugar alcohols such as:
   Glycol(2-carbon)
   Glycerol(3-carbon)
   Erythritol(4-carbon)
   Threitol(4-carbon)
   Arabitol(5-carbon)
   Xylitol(5-carbon)
   Ribitol(5-carbon)
   Mannitol(6-carbon)
   Sorbitol(6-carbon)
   Dulcitol(6-carbon)
   Fucitol(6-carbon)
   Iditol(6-carbon)
   Inositol(6-carbon; a cyclic sugar alcohol)
   Volemitol(7-carbon)
   Isomalt(12-carbon)
   Maltitol(12-carbon)
   Lactitol(12-carbon)
   Polyglycitol; or
b. other Sugars, such as:
   xylitol
   ribose
   fructose
   galactose
   lactose
   maltose
   raffinose
   sucrose.

The first embodiment of the invention is a water-based solution to be applied through iontophoresis. This first embodiment comprises the following ingredients:
2-50 grams of anhydrous dextrose,
0.5-10 grams of aloe vera concentrate (freeze dried 40× powder),
1-20 ml of propylene glycol,
10-100 ml of sterile water, and
1-20 grams of sodium chloride (granular) (or acetic acid).

While the above measurements are ideal, the active ingredients can vary in range from 0.01% to 75% of the total volume of the solution. Among the active ingredients in the first embodiment are anhydrous dextrose, aloe vera concentrate, and propylene glycol.

The second embodiment of the invention is a gel which can be applied directly to the epidermis using an ultra sound machine, and can be absorbed faster than the first embodiment. The second embodiment comprises the following ingredients:
2-50 grams of anhydrous dextrose,
0.5-10 grams of aloe vera concentrate (freeze dried 40× powder),
1-20 ml of propylene glycol,
0.5-5 ml of caprylic/capric triglycerides
10-100 grams of ultrasound gel
0.25-5 ml of simple-gel (Hawkins) gel.

While the above measurements are ideal, the active ingredients can vary in range from 0.01% to 75% of the total volume of the solution. Among the active ingredients in the second embodiment are anhydrous dextrose, aloe vera concentrate, propylene glycol, and caprylic/capric triglycerides.

The third embodiment of the invention is also a gel which can be applied directly to the epidermis without the use an ultra sound machine or iontophoresis, and can be absorbed faster than the first and second embodiment. The third embodiment comprises the following ingredients:
2-50 grams of anhydrous dextrose,
0.5-10 grams of aloe vera concentrate (freeze dried 40× powder),
0.5-5 ml of caprylic/capric triglycerides
1-10 ml of ethoxy diglycol reagent
5-10 grams of a lipoderm base
0.1-5 grams of cetyl myristoleate

The following optional ingredients may be added to the third embodiment:
0.5-5 grams of dimethyl sulfone (or MSM)
0.5-5 ml of pitcher plant extract (1:2 solution), such as Sarapin O.
1-20 ml of distilled water, and
0.5-20 tablets of a homeopathic anti-inflamatory extract, such as Traumeel®

While the above measurements are ideal, the active ingredients can vary in range from 0.01% to 75% of the total volume of the solution. Among the active ingredients in the third embodiment are anhydrous dextrose, aloe vera concentrate, caprylic/capric triglycerides, ethoxy diglycol reagent, lipoderm base, and cetyl myristoleate.

Whenever the following ingredients are used in any of the above three embodiments, the recommended percentage of the solution or get should be as follows:
dextrose at least 5% or 1-50%, preferably 20%, of hypertonic saline
aloe vera concentrate 10%
propylene glycol 10%
caprylic/capric triglycerides 10%
sodium chloride (or acetic acid) 10%
homeopathic anti-inflamatory extract, such as Traumeel® 10%
dimethyl sulfone (or MSM) 10%
cetyl myristoleat 10%
pitcher plant extract, such as Sarapin® 10%.

The three embodiments may be combined with shampoo, soap, or water, without losing their effectiveness.

Although preferred embodiments of the present invention have been shown and described, various modifications and substitutions may be made thereto without departing from the spirit and scope of the invention. Accordingly, it is to be understood that the present invention has been described by way of illustration and not limitation.

What is claimed is:

1. A water-based pain reliever composition applied to the epidermis of mammals comprised of
anhydrous dextrose or hypertonic saline;
aloe vera concentrate;
propylene glycol;
sterile water; and
any one of the following: sodium chloride, acetic acid or hypertonic saline;
all in amounts ranging from 0.01% to 75% of the composition.

2. The pain reliever composition of claim 1, wherein
at least 5% is anhydrous dextrose or 1% to 50% hypertonic saline;
10% is aloe vera concentrate; and
at least 10% is propylene glycol.

3. The pain reliever composition of claim 1, wherein, out of a 100 gram composition, the pain reliever composition comprises:
2-50 grams of anhydrous dextrose or 1-50% hypertonic saline;
0.5-10 grams of aloe vera concentrate constitutes 0.5-10 grams;
1-20 ml of propylene glycol;
10-100 ml of sterile water; and
1-20 grams of sodium chloride or acetic acid.

4. The pain reliever composition of claim 1, wherein anhydrous dextrose is substituted in approximately the same concentrations by sugar alcohols or other sugars.

5. A gel-based pain reliever composition applied to the epidermis of mammals comprised of anhydrous dextrose or hypertonic saline, aloe vera concentrate, propylene glycol, caprylic/capric triglycerides, ultrasound gel, and simple-gel (Hawkins), in amounts ranging from 0.01% to 75% of the composition.

6. The pain reliever composition of claim 5, wherein
at least 5% is anhydrous dextrose or 1-50% is hypertonic saline;
at least 10% is aloe vera concentrate;
at least 10% is propylene glycol, and
at least 10% is caprylic/capric triglycerides.

7. The pain reliever composition of claim 54, wherein, out of a 100 gram composition,
2-50 grams is anhydrous dextrose;
5-10 grams is aloe vera concentrate;
1-20 ml is propylene glycol;
0.5-5 ml is caprylic/capric triglycerides;
10-100 grams is ultrasound gel, and
0.25-5 ml is simple-gel (Hawkins) gel.

8. The pain reliever composition of claim 5, wherein anhydrous dextrose is substituted in approximately the same concentrations by sugar alcohols or other sugars.

9. A gel-based pain reliever composition applied to the epidermis of mammals comprised of anhydrous dextrose, aloe vera concentrate, ethoxy diglycol reagent, caprylic/capric triglycerides, lipoderm base, and cetyl myristoleat, in amounts ranging from 0.01% to 75% of the composition; the lipoderm base being substitutable by any of the following:
lecthicin or other fat soluble granules in the range of 0.001 to 75% (w/w) of the composition;
PLO(Pluronic Lecithin Organogel) in the range of 0.001 to 75% of the composition in gel form;
Urea in the range of 1-50% of the composition;
Oleic acid in the range of 0.001 to 70% of the composition;
Liposomes in the range of 0.01% to 75% of the composition;
Niosomes in the range of 0.01% to 75% of the composition; or
Nanotechnology in the range of 0.01 to 75% chosen from a group consisting of
nanocrystals, liposomes, nanoparticle-protein conjugates, magnetic
nanoparticles, nanogels and biodegradable nanoparticles.

10. The pain reliever composition of claim 9, wherein
at least 5% is anhydrous dextrose or 1-50% is hypertonic saline;
at least 10% is aloe vera concentrate; and
at least 10% is caprylic/capric triglycerides.

11. The pain reliever composition of claim 9, wherein, out of a 100 gram composition,
2-50 grams is anhydrous dextrose;
5-10 grams is aloe vera concentrate constitutes;
0.5-5 ml is caprylic/capric triglycerides;
1-10 ml is ethoxy diglycol reagent;
5-10 grams lipoderm base; and
0.1-5 grams cetyl myristoleat;
Where 5-10 grams lipoderm base can be substituted by any of the following:
5% lecthicin or other fat soluble granules;
30% PLO(Pluronic Lecithin Organogel) in gel form;
10% Urea;
% Oleic acid;
0.01% to 75% Liposomes;
0.01% to 75% Niosomes; or
0.01% to 75% Nanotechnology chosen from a group consisting of nanocrystals, liposomes, nanoparticle-protein conjugates, magnetic nanoparticles, nanogels and biodegradable nanoparticles.

12. The pain reliever composition of claim 9, further comprising at least 10% dimethyl sulfone or Methylsulfonylmethane (MSM), at least 10% pitcher plant extract, distilled water, and a homeopathic anti-inflammatory extract.

13. The pain reliever composition of claim 11, further comprising
0.5-5 grams of dimethyl sulfone Methylsulfonylmethane (MSM);
0.5-5 ml of pitcher plant extract (1:2 solution);
1-20 ml of distilled water; and
0.5-20 tablets of a homeopathic anti-inflammatory extract.

14. The pain reliever composition of claim 11, wherein anhydrous dextrose is substituted in approximately the same concentrations by sugar alcohols or other sugars.
